(19) United States
(12) Patent Application Publication
(10) Pub. No.: US 2006/0135608 A1
(43) Pub. Date: Jun. 22, 2006

(54) Eicosapentaenoic acid (epa) for treating anorexia nervosa (an) and bulimia

(76) Inventors: David Frederick Horrobin, Stirling (GB); Agnes Ayton, Staffordshire (GB); Sherri Clarkson, Scotland (GB)

Correspondence Address: JACOBSON HOLMAN PLLC, 400 SEVENTH STREET N.W., SUITE 600, WASHINGTON, DC, 20004, US

(21) Appl. No.: 10/528,114

(22) PCT Filed: Sep. 16, 2003
(86) PCT No.: PCT/GB03/03985

§ 371 Date: Jun. 16, 2005

(30) Foreign Application Priority Data
Sep. 16, 2002 (GB) 0221480.7

(51) Int. Cl. A61K 31/202

(52) U.S. Cl. 514/547; 514/560

(57) ABSTRACT

Eicosapentaenoic acid (EPA) is used in the treatment of anorexia nervosa, bulimia and related clinical syndromes.

Anorexia nervosa (AN) is a severe illness which particularly effects adolescent girls and young women, but which can occur in both males and females of any age. There is a fear of weight gain, coupled with a pathological need to lose weight. Sufferers usually have a disturbed body image which means that they always perceive themselves as much heavier and fatter than they really are.

AN is becoming more and more common. AN sufferers often become strong advocates for the idea of weight control and do all they can to persuade others follow the same path. There are now large numbers of “PRO-ANA” web sites which promote AN and describe in great detail methods to enhance weight loss. These include, of course, strict dieting, methods of deceiving others about how much is being eaten, using diuretic drugs to promote water loss, using laxatives to provide diarrhoea, and using emetic drugs and other techniques to promote vomiting. In variants of the basic AN syndrome, some individuals eat relatively normally, or even binge eat large amounts, followed by vomiting and other extreme techniques to get rid of the food. This variant of AN is known as bulimia.

Although there are thousands of different theories, the root cause of AN remains unknown. No treatment has ever been found to be consistently successful. A recent detailed prospective study of available treatments found that there was no relationship between the type of treatment used and any long-term outcome (D I Ben-Tovim et al. Outcome in patients with eating disorders: A five-year study. Lancet, 2001; 357: 1254-7). This means that no treatment is effective and probably also means that most of the theories on which treatments are based are wrong.

Those who do not know much about AN frequently underestimate its seriousness. In fact more than half of all patients never properly recover and have some form of lifelong eating disorder which seriously disrupts their lives. About 20% of sufferers will die, by far the highest death rate in any relatively common disease which affects young women, and which apparently starts in a way which is relatively benign, the need to diet.

New treatments are therefore urgently required. The present inventors claim a new treatment, the use of eicosapentaenoic acid (EPA) or one of its derivatives for the management of AN or related disorders such as bulimia. EPA is a highly unsaturated essential fatty acid which has been found useful in psychiatric and neurological disorders (EP 1148873 and EP 0956013). However, it has never, to the knowledge of the applicant, been proposed as a treatment for AN or bulimia. Indeed, in view of the unsatisfactory outcomes obtained when using psychiatric drugs for AN, there is no reason to believe on the basis of prior art that AN might respond to EPA.

The present invention provides a method of treating anorexia nervosa, bulimia and related clinical syndromes by administering to a subject eicosapentaenoic acid (EPA) in any appropriate form which can be assimilated by the body. The subject may one showing symptoms of, or believed to be at risk from AN or a related syndrome. The present invention also provides use of eicosapentaenoic acid (EPA) in any appropriate form which can be assimilated by the body in the manufacture of a medicament for the treatment of anorexia nervosa, bulimia and related clinical syndromes.

Eicosapentaenoic acid (EPA) can be administered in many different forms. The abbreviation “EPA” is used herein to refer to the acid, or its derivative, which is used in the preparations employed in the present invention. Thus the forms of EPA used in the present invention include the free acid, salts such as those of sodium, potassium, lithium or any other appropriate salt, mono-, di-, or triglycerides, phospholipids of various sorts, amides, esters including ethyl, methyl or other esters, and any other derivative which is biologically compatible and which can be demonstrated by standard assay techniques to raise the level of EPA in the blood of the patient. Combinations may be used. Preferred are the triglyceride or ethyl ester, the ethyl ester being particularly preferred.

EPA can be synthesised but with great difficulty because of its thirty-two isomers, only one of which involves all the double bonds in the cis configuration and which is biologically active. It is usually therefore prepared from natural EPA-containing sources including micro algae and other micro-organisms, a wide range of different marine oils from fish, shellfish and marine mammals and, increasingly, from genetically modified micro-organisms or higher plants. EPA from any of these sources may be used in the invention. These provide sources of the acid and its derivatives.

The EPA may be used in the form of the natural oils or preferably in partially purified or fully purified extracts or semi-synthetic derivatives containing preferably more than 70% of the pure compound (the free acid and/or its derivatives) and very preferably more than 90% or more than 95% of the pure compound. Pure EPA-triglyceride or the pure ethyl ester of EPA are particularly suitable for these purposes. It is increasingly evident that EPA binds to highly specific sites in cells and that the binding can be interfered with by other fatty acids which can thus interfere with the activity of the EPA itself (D F Horrobin, Progr Drug Res, 2002). The best therapeutic results will therefore be obtained when the final pharmaceutical dosage form contains less than 10% in total and less than 3% individually of other fatty acids which might interfere with the action of EPA. Preferably the final dosage form should contain less than 5% in total and less than 2% individually of other fatty acids which might interfere with the action of EPA. The fatty acid of most concern in this context is the related fatty acid docosahexaenoic acid (DHA). Other fatty acids to be taken into consideration in this calculation are linoleic acid (LA) and arachidonic acid (AA). Preferably, the EPA contains less than 10% in aggregate and less than 3% individually of docosahexaenoic acid, linoleic acid and arachidonic acid. Still preferably, the EPA contains less than 5% in aggregate and less than 2% individually of docosahexaenoic acid and linoleic acid. It may also be preferred that there is less than 2% arachidonic acid in the EPA. EPA preparations of 1% or less DHA, LA or AA may be used. Alternatively, an EPA preparation in which DHA is substantially absent may be employed. In addition, the preparation may be substantially free from LA or AA, or both LA and AA.

The total dose of EPA to be used daily in the treatment of AN and related conditions may range from 50 mg to 20 g per day but will usually be in the range of 100 mg to 5 g/day and particularly in the range 300 mg to 3 g/day.

The usual route of administration will be in a pharmaceutical dosage form of capsules or micro-capsules or other appropriate form prepared by those skilled in the art. Other appropriate formats, particularly for AN patients, are:
1. Any form of liquid or emulsion or related dosage form for oral administration.
2. Any form of preparation for parenteral administration by intramuscular or intravenous routes which may be needed to bypass the food phobias seen with AN patients.
3. The addition of EPA at the appropriate dose to specialist medical foods which are specifically used for the treatment of AN patients, particularly liquid foods for oral administration or for administration by enteral tube feeding. EPA may also be added to nutritional supplements for patient with AN or related disorders, to be administered intravenously.

### EXAMPLES

### Example 1

A 15-year-old patient presented with a 14-month history of dieting and eating difficulties. These had started with dietary restrictions and excessive exercise and proceeded to laxative abuse. Two months prior to being first seen she had stopped taking all solid food. When first seen her weight was still within the normal range for her height at 55 kg for 1.63 m. However, she had lost 8 kg since stopped solid food, had stopped menstruation and begun to grow the fine, downy “lanugo” hair over her body which is common in AN.

She was treated with a standard AN regime of family therapy, psychotherapy and dietary advice. This was ineffective and over the next two months she lost around 10 kg which necessitated her admission to hospital. At this point she was extremely distressed and unable or unwilling to maintain a conversation. Despite her emaciation she was still preoccupied with being fat and wanted to lose more weight. Her heart rate was very slow and her blood glucose was low, signs of starvation. She was treated as an emergency with compulsory naso-gastric feeding with parental consent. After two weeks of this therapy she had gained a little over 2 kg and begun to eat small amounts by mouth. At the end of this time her family removed her from hospital against medical advice.

Over the following ten days she lost a further 5 kg in weight to 42 kg. Her doctors believed that her life was in danger and so obtained an order for compulsory admission to hospital. At the start of this admission she was treated with 1 g/d of ethyl-eicosapentaenoate (E-EPA). This transformed her response to treatment. Over the following weeks she began to eat normally and within 12 weeks she was back to 57 kg. Her mood and cognitive functions improved and she became normally communicative. Instead of being obsessed by weight and food to the exclusion of everything else, she became interested in all aspects of her life and her future. She lost her distorted body image perceptions and became confident about her appearance. After 12 weeks she was discharged from hospital and her body weight stabilised around a normal 62-65 kg. She took a summer job which she enjoyed and completed successfully and enrolled in a college course. The changes with time are summarised in table 1.

TABLE 1
Changes in the status of a patient with AN treated
with ethyl-EPA. The Morgan-Russell (MR) Outcome Scale is a
well-recognised scale for assessing the status of patients
with AN. The overall scale (MR-O) addresses the whole
picture, while sub-scales address issues like food intake (MR-
A), mental state (MR-C) and overall social-economic-health
state (MR-E) . The overall scale and its sub-scales are all
scored from 0 to 12 where 0 indicates a severe problem and 12
indicates completely normal.

| Event | Wt kg | MR-O | MR-A | MR-C | MR-E |
|---|---|---|---|---|---|
| Pre-illness | 63 | 12.0 | 12.0 | 12.0 | 12.0 |
| 1^{st} doctor visit | 55 | 1.9 | 2.7 | 4.0 | 1.0 |
| 1^{st} hospital admission | 45 | 1.9 | 2.7 | 4.0 | 0.0 |
| 1^{st} hospital discharge | 47 | 1.0 | 0.0 | 4.0 | 0.0 |
| 2^{nd} hospital admission | 42 | 1.2 | 0.0 | 4.0 | 1.0 |
| 2^{nd} hospital discharge | 57 | 12.0 | 12.0 | 8.0 | 9.0 |
| on EPA | | | | | |
| 3 months after discharge | 63 | 12.0 | 12.0 | 12.0 | 11.0 |

### Example 2

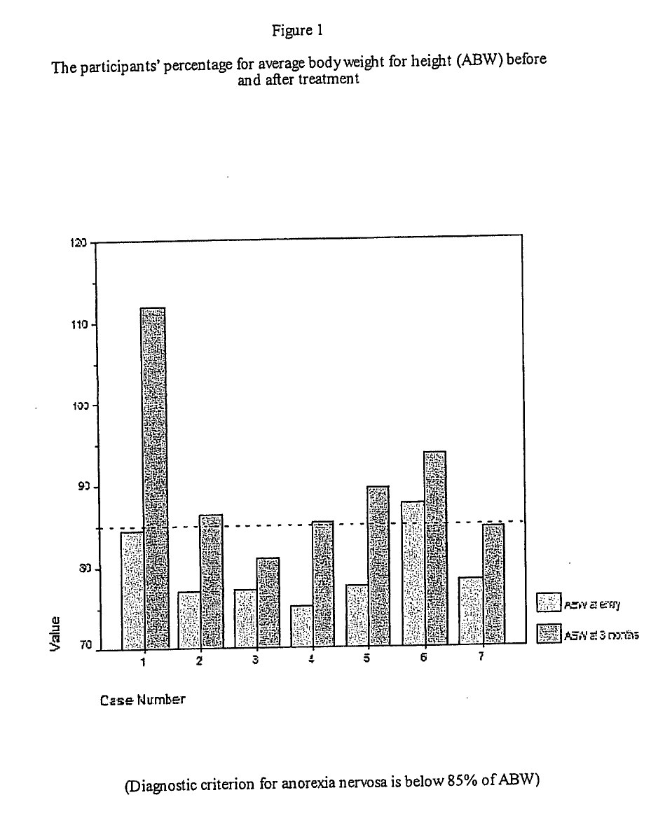

Seven patients underwent treatment of their disorders using EPA. FIGS. 1-5 summarise the results of this study. Participants were given 1 g/day ethyl-EPA (E-EPA) for an initial 3 month period. The E-EPA provided by Laxdale Limited was over 95% pure EPA. If the patient and family wished to continue beyond 3 months, the dose was continued, and in some cases increased beyond 1 g/day. All patients were offered the standard treatment available at the local district health services, including full psychiatric and physical assessment, regular monitoring of physical parameters. Parameters monitored on a monthly basis included the patient's weight and height. BMI, and average body weight and height (ABW) were calculated using Weight 4 Height software (based on 1990 British reference data by the Child Growth Foundation). The following standard psychometric measures were used: EDI-2, BDI-2, CGAS, CGI-S, Morgan-Russell, and patient Likert Scales (including problems, general and improvement).

FIG. 1 shows the participants' average body weight percentage before and after treatment;

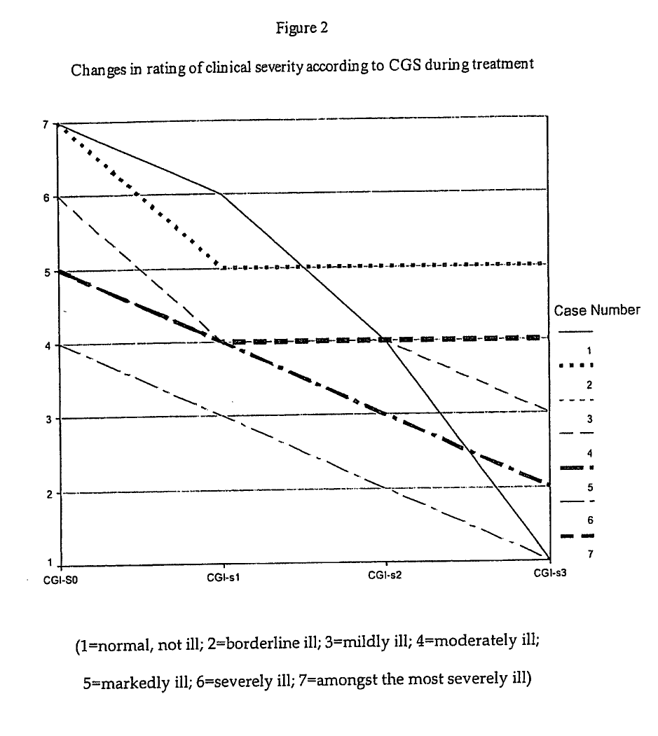

FIG. 2 shows changes in rating of clinical severity according to CGI-S (Clinical Global Impressions scale for Severity) during treatment

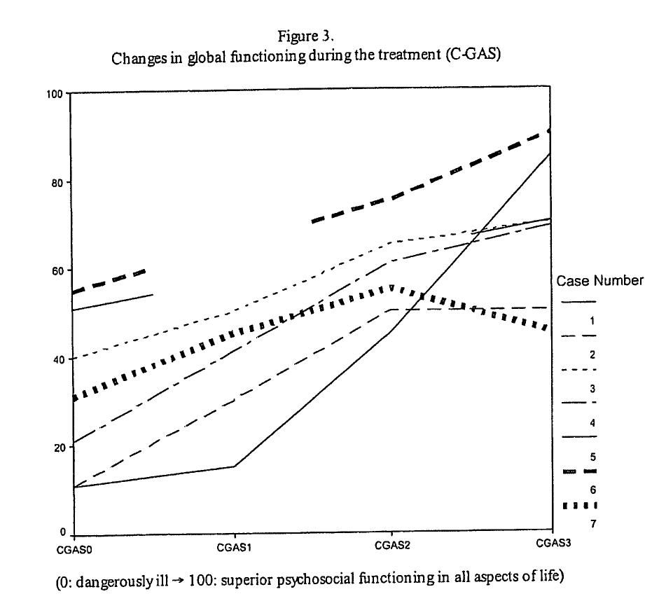

FIG. 3 shows changes in global functioning (C-GAS) during treatment

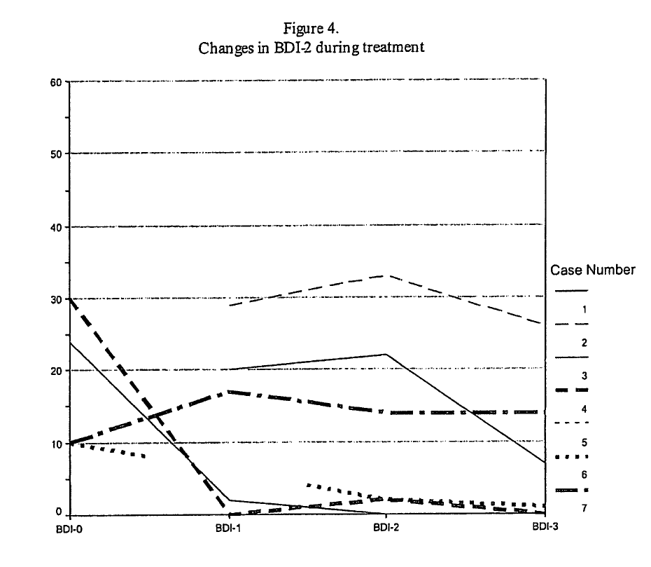

FIG. 4 shows changes in BDI-2 (Beck Depression Inventory) during treatment

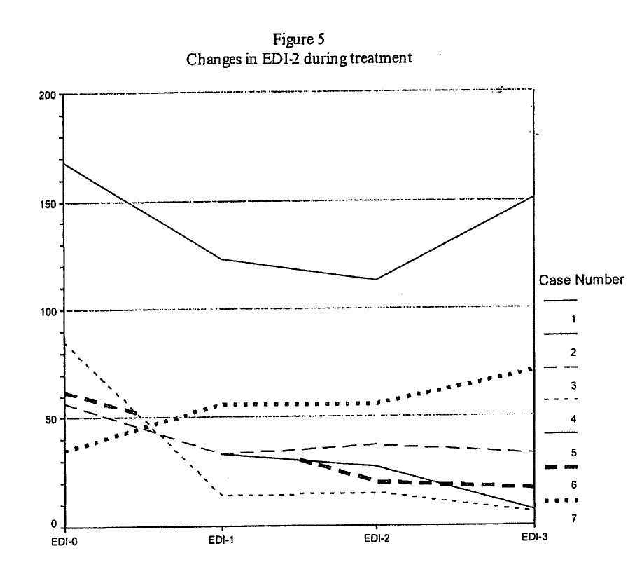

FIG. 5 shows changes in EDI-2 (Eating Disorder Inventory) during treatment

Patient No 1

Patient No 1 was 15.6 years old when she started ethyl-EPA treatment. She had an 18-month history of restrictive anorexia, which arose in the context of sexual abuse and bullying. There was a family history of polycystic ovary syndrome(POS), obesity and depression. During the last four months of her illness, her condition rapidly deteriorated and she lost about ⅓ of her body weight (pre-morbid BMI was above 24). She had secondary amenorrhoea, poor circulation and lanugo. Blood tests revealed hypoglycaemia, leucopenia and abnormal LFTs. By the time she was admitted to hospital her BMI was 16.9 (ABW 83.6%). Her mental state was severely impaired, she was hardly accessible, she was overwhelmingly anxious and had severe body image distortion. She was started on 1 g ethyl-EPA (E-EPA) at a purity of over 95% EPA a few weeks after commencing nasogastric re-feeding. In addition, she also received Forceval 2 caps/day and Solvazinc, to correct micronutrient deficiencies. She was so unwell mentally that she was unable to complete the baseline psychometric measures. The nasogastric feeding stopped after 3 weeks, as she was prematurely discharged from hospital against medical advice. She continued to lose weight rapidly, and as it was not possible to ensure treatment on a voluntary basis, she was eventually detained under Section 3 of the Mental Health Act. Afterwards, her treatment continued on the general adolescent mental health unit, and she received oral re-feeding and milieu therapy. She was unwilling to engage in individual psychotherapy, and repeated attempts of family therapy failed. However, both parents and the patient were willing to continue with the E-EPA treatment. There was a remarkable improvement after 2 months of treatment, which included improved appetite, mood, self-esteem, interest in her future and normalisation of the psychometric measures. She developed acne, which later was found to be the consequence of POS. The patient completed three months of E-EPA treatment, but decided to stop afterwards, as she was concerned about ongoing weight gain (BMI 22.8, ABW 111%). She returned to college and her level of functioning was higher than pre-morbidly for about three months after the completion of the E-EPA treatment. However, after about 6 months, her mood deteriorated and she experienced significant mood swings. At one-year follow-up, she was approximately her pre-morbid weight; there was no return of her anorexia, and she did not develop bulimic symptoms, despite the significant psychosocial stressors in her life. She was sexually active and her periods returned.

Patient 2

Patient 2 was 14.5 years old with two years' history of restrictive diet, excessive exercise and primary amenorrhoea. She suffered from chronic low self-esteem and low mood. There was a family history of depression. There was no clear precipitating event before the anorexia. She was admitted to the paediatric intensive care unit as a medical emergency and had to be resuscitated on admission to hospital due to hypoglycaemia and cardiovascular collapse. At that point, her BMI was 14.4, ABW: 76.3%. She demonstrated a high level of psychopathology, including severe body image distortion, extreme fear of food, a desire to lose further weight even if it meant losing her life, and obsessive symptoms. Initial blood tests showed somewhat increased cholesterol, bilirubin and increased amino transferase, and low levels of zinc and selenium. This patient had consistently low zinc levels despite supplementation. She was nasogastrically (NG) fed on parental consent until her physical parameters stabilised, and she reached BMI 16.1 (ABW: 84.4%). The E-EPA treatment started when she was on the NG feed. 1 g/day was administered of over 95% pure EPA. Following the discharge from the paediatric ward, her parents only consented to day hospital treatment on the adolescent mental health unit. They refused family therapy, but she accepted individual psychotherapy, which was based on motivational and psycho-educational principles. As her depressive and obsessive symptoms remained pervasive, antidepressant treatment was offered, but again the parents did not consent to this. She had partially improved by three months (ABW 86.34% and there was only small improvement in her psychometric measures). She was discharged by her parents prematurely, but maintained weight for a further three months. She stopped the E-EPA treatment after 6 months and this resulted in a significant downturn, both in terms of weight (lowest ABW 73%) and psychopathology. The parents refused readmission into hospital, but agreed to restart the E-EPA and zinc treatment. This was followed by significant improvement. In the later stages of the treatment, the E-EPA dosage was increased to 2 g/day. At 1-year follow-up, her BMI was 17.74 (ABW: 88.3%), she had much improved psychosocial functioning, improved social life, but no boyfriend. She remained amenorrhoeic.

Patient 3

This 13.3 year old female patient was referred to the adolescent unit with three years' history of restrictive diet, primary amenorrhoea, growth retardation, and delay in sexual development. She was pre-pubertal. The patient denied body-image distortion and there were significant emotional problems and low mood. Food Avoidance Emotional Diagnosis was made (which is equivalent to atypical anorexia nervosa). Her BMI at the point of referral was 13.3 (ABW 74.4%). Despite her low body weight, she was physically stable, and she was managed as a 5-day/week inpatient on the adolescent mental health unit. She received oral re-feeding, milieu therapy, psycho-education and supportive counselling. She had low ferritin and low folate level, which were corrected. She did not engage in psychotherapy, and the family did not participate in family therapy due to difficulties with transport. She received 1 g/day over 95% pure E-EPA treatment and grew 3 cm in three months whilst receiving E-EPA, her BMI became 15.5 (ABW: 81%) and her puberty began. Her mental state significantly improved and she became cheerful and positive. She had no abnormal preoccupation with food and was able to consume a wide range of high calorific foods. Unfortunately, after her discharge from the adolescent unit, the parents regularly missed follow-up appointments, and her compliance with E-EPA declined. At 6 months, she was the same weight as at discharge, and there was no further growth. She was lost to follow-up after 6 months.

Patient 4

This 14.5 year old female was referred urgently with 6-months' history of restrictive diet and rapid weight loss, in the context of bullying and family problems. She had three months history of amenorrhoea. She was unable to eat. On physical examination, her BMI was 14.8 (ABW: 74.7%). She had bradycardia, low blood pressure, and poor peripheral circulation. She was cachectic, she had dry skin and she was constipated. Mental state examination revealed low mood, severe body-image distortion, preoccupation with weight and shape, and obsessive behaviour around food. On the paediatric ward she was NG-fed, and received Solvazinc to correct zinc deficiency. Following her discharge from the paediatric hospital a few weeks later (at ABW 83.9%), the family only agreed to minimal mental health input. However, she was willing to continue with the 1 g/day over 95% pure E-EPA treatment and the E-EPA was administered for a total of 6 months. There was a dramatic improvement in her mood after two months, and a marked improvement in the psychometric measures. She resumed an active social life, and became interested in boyfriends. Her weight stabilised around 85.5% ABW. However, her weight deteriorated within three months after she stopped taking the E-EPA (80% ABW). Her periods had not returned by the end of the year.

Patient 5

Patient 5 volunteered her participation in the study. She was a 22 year old pharmacology graduate with 7 years' history of anorexia nervosa, with bulimic symptoms. There was a family history of depression. She had no previous admission despite the fact that her lowest BMI was around 14.15, due to lack of local care services. She was administered E-EPA from a source different to the present inventors and offered to keep in touch and advise of the effects. She had secondary amenorrhoea, but was sexually active. She had low self-esteem, poor impulse control and significant co-morbid anxiety with panic attacks. As she was not under the care of the local services, she received no psychological treatment, apart from one psycho-educational session. Her BMI before starting the 1 g/day E-EPA was 17.15 (ABW: 77%). There was a dramatic improvement after three months in terms of her weight (BMI 20, ABW: 90%), eating habits and mood, but her anxiety did not improve. The E-EPA was increased to 4 g/day and this helped with her panic attacks. She was sexually active and happy and 6 months follow-up.

Patient 6

A 17-year old male presented with 9 years' history of dietary restriction and preoccupation with weight and shape. He became highly obsessional around food, which caused significant arguments at home and impacted on his social life. On the first presentation, his BMI was 17.57 (ABW: 87%). His height was on the 0.01 centile, suggesting severe growth retardation (there was no growth hormone deficiency) and delay in sexual development. There was little evidence of puberty, he had no facial hair, his voice was not broken, and he had the appearance of a much younger child. He had low blood pressure, mild bradycardia, and poor peripheral circulation. There was a lack of libido. The patient and family wanted outpatient treatment, and because of his schedule (he was repeatedly out of the area for several weeks) he received only psycho-education and dietary counselling. He improved dramatically within the first 4-6 weeks of 1 g/day over 95% pure E-EPA treatment. By the end of three months, his BMI was 19.1 (ABW: 93.6%), he grew 3 cm, there was a complete resolution of his anorexic symptoms and his libido returned. The only residual symptom at 6 months was mild anxiety.

Patient 7

This 13.5 year old female patient presented with 18 months' history of dietary restraint and excessive exercise, growth and developmental delay. She was pre-pubertal. There was a family history of anorexia and depression, and major family problems. She had low mood, preoccupation with weight and shape and body-image distortion. As she was physically stable, she was admitted to the adolescent mental health unit as w 5-day/week inpatient. She received oral re-feeding, milieu therapy, family therapy and individual therapy. Her BMI before starting 1 d/day over 95% pure E-EPA treatment was 14.8 (ABW: 78.21) and at the end of three months treatment with E-EPA it was 16.21 (ABW 84.5%). She grew 1.5 cm during these three months. She had an emotional downturn at about 6 weeks. This was in response to parental separation, impending divorce and moving house. There is no follow-up information on this patient.

It is remarkable that no patient deteriorated whilst taking the E-EPA. In contrast, a patient who delayed participation in the study for 6 months deteriorated during this time. Of the seven patients discussed in Example 2, there was partial improvement in four cases, and complete recovery in three cases. Those patients who had growth delay responded with significant growth during the time of the E-EPA treatment. The recruitment and adherence to the treatment was good, given that the majority of patients were reluctant to engage in standard treatment for anorexia nervosa, including individual therapy and family therapy.

These dramatic responses to treatment demonstrate an entirely novel and unexpected approach to the management of AN and related eating and vomiting disorders. The invention is therefore directed to the use of EPA in any appropriate dosage form for the management of these disorders. Since patients with AN often suffer from general micronutrient deficiencies it is appropriate to combine the EPA with micronutrient supplements either provided separately or in the same dosage form. Example supplements are zinc supplements, for example Solvazinc™, and Forceval™. Appropriate dosage forms for the EPA include pharmaceutical unit dosage, nutritional supplements and specialist foods, including foods for administration by naso-gastric tubes or other enteral or parenteral routes.

1. A method of treating anorexia nervosa, bulimia and related clinical syndromes by administering eicosapentaenoic acid (EPA) in any appropriate form which can be assimilated by the body.

2. Use of eicosapentaenoic acid (EPA) in any appropriate form which can be assimilated by the body in the manufacture of a medicament for the treatment of anorexia nervosa, bulimia and related clinical syndromes.

3. A method according to claim 1, in which the EPA is from a natural EPA-containing oil.

4. A method according to claim 1, in which the EPA is in the form of the free acid, an appropriate salt, a mono-, di-, or triglyceride, a phospholipid, an amide, an ester or any other biologically compatible derivative.

5. A method according to claim 1, in which the EPA is in the form of the triglyceride or the ethyl ester.

6. A method or use according to claim 1, in which the EPA is more than 70%, preferably more than 90% and very preferably more than 95% pure.

7. A method or use according to claim 6, in which the EPA contains less than 10% in aggregate and less than 3% individually of docosahexaenoic acid, linoleic acid and arachidonic acid.

8. A method or use according to claim 6, in which the EPA contains less than 5% in aggregate and less than 2% individually of docosahexaenoic acid and linoleic acid.

9. A method or use according to claim 7, in which the EPA is in the form of the ethyl ester.

10. A method or use according to claim 1, in which the EPA is for oral administration in an appropriate pharmaceutical dosage form and is given at a dose between 50 mg and 20 g/d, preferably between 100 mg and 5 g/day and very preferably between 300 mg and 3 g/day.

11. A method or use according to claim 1, in which the EPA is for parenteral, intramuscular or intravenous administration in an appropriate pharmaceutical dosage form.

12. A method or use according to claim 1 wherein the EPA is added to a nutritional supplement for patient with AN or related disorders, such supplement to be taken orally, or given by enteral tube, or given intravenously.
